(19) United States
(12) Patent Application Publication
(10) Pub. No.: US 2011/0130629 A1
(43) Pub. Date: Jun. 2, 2011

(54) ENDOSCOPE COVER FIXING DEVICE AND FIXING SYSTEM

(75) Inventors: Motonori Watanabe, Fukuroi-shi (JP); Keita Iwamizu, Fukuroi-shi (JP)

(73) Assignee: TYCO HEALTHCARE GROUP LP, Mansfield, MA (US)

(21) Appl. No.: 12/953,639

(22) Filed: Nov. 24, 2010

(30) Foreign Application Priority Data
Nov. 30, 2009 (JP) 2009271766

(51) Int. Cl. A61B 1/00

(52) U.S. Cl. 600/125

(57) ABSTRACT

An endoscope cover fixing device to reliably fix a cover to an endoscope without associated damage. The endoscope cover fixing device includes a fixing device main body and a pair of fixing protrusion portions. The fixing device main body includes a curved portion and insert holes, and is deformable in the direction in which the bend amount increases. The pair of fixing protrusion portions, which have contact surfaces, are provided to protrude from a concave surface side of the fixing device main body and are disposed in two opposing regions of the concave surface. The contact surfaces are disposed in parallel with a fixed distance therebetween. During deformation of the fixing device main body, the pair of fixing protrusion portions move in parallel in opposing directions. As a result, the contact surfaces contact opposing positions on the outer circumferential surface of the endoscope by way of the cover.

### FIELD OF THE INVENTION

The present invention generally relates to an endoscope cover fixing device for, when a cover having a closed distal end is placed over an endoscope, fixing the position of the cover on the endoscope, and to a system employed for fixing the cover on the endoscope.

### BACKGROUND OF THE INVENTION

By necessity, medicine requires the diagnosis and treatment of interior body cavities that are not otherwise visible, and endoscopes are a well-known conventional medical instrument for implementing such diagnoses in a comparatively simple manner. Common endoscope structures comprise a lens portion provided in the distal end of a tube of a size that is insertable in an interior body cavity, and images captured by this lens portion are transmitted by way of optical fibers arranged within the tube.

Viscous body fluids and impurities affix to the outer circumferential surface of an endoscope during use. Accordingly, endoscopes must be thoroughly cleansed and disinfected after use to prevent the transfer of infectious diseases and the like. However, the complicated and time-consuming nature of the endoscope cleansing and disinfecting operations affects the operability thereof. A further problem pertains to the accelerated deterioration of the endoscope caused by the regular cleansing and disinfecting thereof.

With this in mind, the use of a sheath-like, contamination-preventing endoscope cover which has a closed distal end and which is placed over the endoscope to prevent contamination of the endoscope and is discarded after use has been recently considered.

However, the acquisition of satisfactory images with a cover placed over the endoscope requires that the cover be fitted closely to the lens portion in the distal end of the tube. However, with covers of this type, dimensional variations in the manufacturing process thereof are unavoidable. Such variations often render it difficult to fit the the cover closely to the lens portion of the tube.

With this in mind, structures for fixing a cover to an endoscope have been hitherto proposed (for example, see Japanese Patent No. 335932). In the cover-type endoscope as described in the '932 Japanese patent, an engagement portion is provided in the inner circumference of the proximal end side of the cover, and a slidable latch portion corresponding to the engagement portion is provided in the outer circumference of an operating portion of the endoscope. According to this configuration, friction between the latch portion and the engagement portion causes the latch portion to engage closely with the engagement portion which, in turn, facilitates fixing of the position of the cover on the endoscope.

However, the close engagement and fixing of a cover to an endoscope necessitates the employment of components that enhance the rigidity of the cover itself, and that increase the friction force in the conventional art as described above. The employment of these components increases the complexity of the machining and the assembly of the device, and also increases manufacturing and other costs.

Thereupon, the employment of a fixing device for positioning and fixing a cover to an endoscope configured separately to the endoscope and the cover such as a conventional medical clamp for example, was briefly considered. However, the employment of a conventional medical clamp comprises clamping the cover and the endoscope together with a pair of clamping portions and locking them in the clamped state. However, there is a significant pressing force or pressure exerted on the endoscope in the radial direction. This increases the likelihood that the image guide or light guide of the endoscope, usually constituted from optical fibers, will be damaged.

### SUMMARY OF THE INVENTION

With the foregoing conditions in mind, it is an object of the present invention to provide an endoscope cover fixing device and fixing system able to reliably fix the position of a cover onto an endoscope without associated endoscope damage.

In general, this invention is directed to an endoscope cover fixing device employed for, when a cover having a closed distal end is placed over an endoscope, fixing the position of the cover on the endoscope. The device comprises a fixing device main body that has a bent section deformable in the direction in which the bend amount thereof increases. The device further comprises an insert portion into which the endoscope is insertable. The device further comprises a pair of fixing protrusion portions that have a contact surface and that are provided to protrude from a concave surface side of the aforementioned fixing device main body. The pair of fixing protrusion portions are mutually oppositely disposed in two opposing regions on the two sides of the concave surface. The contact surfaces of the pair of fixing protrusion portions are disposed in a parallel positional relationship with a constant distance therebetween and in a parallel positional relationship to the longitudinal direction of the endoscope when the endoscope is inserted in the insert portion. The pair of protrusion portions move in parallel in opposing directions during the deformation of the fixing device main body to ensure that the contact surfaces, by way of the cover, contact opposing positions on the outer circumferential surface of the endoscope.

When deformation occurs in the direction in which the bend amount of the fixing device main body increases, the pair of (oppositely disposed) fixing protrusion portions move in parallel, opposing directions. At this time, the contact surfaces of the pair of protrusion portions, with the gap distance therebetween maintained constant, contact opposing positions on the outer circumferential surface of the endoscope by way of the cover. Accordingly, during the fixing operation, a constant pressing force is exerted in the radial direction of the endoscope, and the exertion of an excess pressing force thereon is avoided. As a result, the cover is able to be reliably fixed in position on the endoscope without associated endoscope damage.

According to an embodiment of the invention, the endoscope cover fixing device is characterized in that the region across which the contact surfaces contact the outer circumferential surface of the endoscope by way of the cover extends linearly along the longitudinal direction of the aforementioned endoscope. The length of these regions of contact are established to be larger than the pitch of a coil disposed in an over-tube of the endoscope. Because the contact region of the contact surfaces extends lengthwise along the longitudinal direction of the endoscope, the pressing force is dispersed rather than concentrated on a specific region. As a result, damage to the endoscope is able to be more reliably prevented.

According to another embodiment of the invention, the endoscope cover fixing device is characterized by the provision of two open ends in the aforementioned fixing device main body, the provision in the two open ends of latch portions which latch together to maintain a deformed state, and the provision of a contact surface distance-maintaining mechanism for preventing the distance between the contact surfaces from increasing as a result of the displacement of the pair of fixing protrusion portions in the deformed state. The deformed state is maintained by the latch portions, and an increase in the distance between the contact surfaces is prevented by the contact surface distance-maintaining mechanism. Accordingly, a constant pressing force is maintained during the positional fixing operation and, as a result, the cover is able to be even more reliably positionally fixed to the endoscope.

Aspects of the invention are further directed to an endoscope cover fixing system for, when a cover having a closed distal end is placed over an endoscope, employing a fixing device to fix the position of the cover on the endoscope. The endoscope cover fixing system comprises a pair of fixing protrusion portions that have contact surfaces and a parallel positional relationship. The contact surfaces contact opposing positions on the outer circumferential surface of the endoscope by way of the cover with a constant distance maintained between the contact surfaces. As a result, a constant pressing force is exerted during the fixing operation while the position of the cover is fixed. Because the contact surfaces contact opposing positions on the outer circumferential surface of the endoscope by way of the cover with a constant distance maintained between the contact surfaces, a constant pressing force is exerted in the radial direction of the endoscope during the fixing operation, and the exertion of an excessive pressing force thereon is avoided.

Accordingly, the position of the cover is reliably fixed to the endoscope without associated endoscope damage.

Other objects and features will be in part apparent and in part pointed out hereinafter.

### BRIEF DESCRIPTION OF THE DRAWINGS

FIG. 1 is a schematic diagram of an endoscope cover fixing device, an endoscope and a cover of a first embodiment of the present invention;

FIG. 2(a) is a front view of the endoscope cover fixing device of FIG. 1 in an open state;

FIG. 2(b) is a cross-sectional view taken along the line A-A of FIG. 2(a);

FIG. 3(a) is a left-side view of the endoscope cover fixing device (open state) of FIG. 2(a);

FIG. 3(b) is a right-side view of the endoscope cover fixing device (open state) of FIG. 2(a);

FIG. 4(a) is a front view of the endoscope cover fixing device of FIG. 1 in a closed state;

FIG. 4(b) is a cross-sectional view along the line B-B of FIG. 4(a);

FIG. 5(a) is an enlarged, fragmentary cross-sectional view of the endoscope cover fixing device (open state) of FIG. 2(a) with the position of the cover fixed on the endoscope;

FIG. 5(b) is an enlarged, fragmentary cross-sectional view of the endoscope cover fixing device (closed state) of FIG. 4(a);

FIG. 6(a) is a front view of the endoscope cover fixing device (open state) of another embodiment of the present invention;

FIG. 6(b) is a right-side view of the endoscope cover fixing device (open state) of FIG. 6(a); and

FIG. 7 is a cross-sectional view of an endoscope cover fixing device (open state) of yet another embodiment of the present invention.

### REFERENCE CHARACTERS

11 Endoscope Set

21 Cover

22 Distal end of cover-tube

23 Proximal end of cover-tube

31 Endoscope

32 Cover-tube

33 Coil

41,81,91 Endoscope cover fixing device

42 Fixing device main body

43 Curved portion constituting bent section

44 First arm portion

45 Second arm portion

46 (First) open end

47 (Second) open end

51 Curved portion

52 Tubular portion

53 Latch release portion

54,57 Insert hole constituting insert portion

55 Concave surface

61,62 Fixing protrusion portion

61a,62a Contact surfaces

65a (First) latch portion

65b (Second) latch portion

82 Groove from which contact surface distance-maintaining mechanism is constituted

83 Protrusion from which contact surface distance-maintaining mechanism is constituted

92,93 Protruding piece from which contact surface distance-maintaining mechanism is constituted

C1 Longitudinal direction of endoscope

L1 Distance

P1 Pitch

R1 (Contact) region

Corresponding reference characters indicate corresponding parts throughout the drawings.

### DESCRIPTION OF THE PREFERRED EMBODIMENTS

An endoscope cover fixing device able to reliably fix the position of a cover on an endoscope without associated endoscope damage is provided.

### First Embodiment

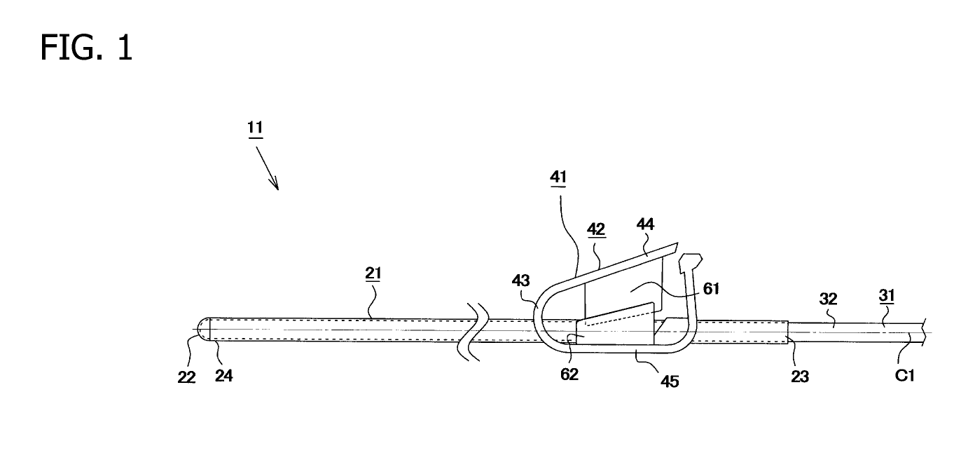

An endoscope cover fixing device of a first embodiment of the present invention will be hereinafter described with reference to FIGS. 1 to 5.

FIG. 1 schematically shows an endoscope set 11 comprising an endoscope cover fixing device 41, an endoscope 31 and an endoscope cover 21 of the first embodiment. The endoscope 31 comprises a lens portion (not shown) provided at a distal end 22 of a flexible cover tube 32. Tube 32 has a diameter suitable for insertion into a body cavity interior. In a non-limiting embodiment, the endoscope 31 has a maximum diameter of 0.5 mm, and is generally on the order of 0.4 mm. The endoscope cover 21 constitutes a sheath-like member formed from a flexible and light-permeable material such as a synthetic resin, and has a slightly larger internal diameter than the diameter of the endoscope 31. The distal end 22 of the endoscope cover 21 is rounded and closed in the illustrated embodiment. The cover 21 is placed over the endoscope 31 and, in this state, the inner surface of the distal end 22 of the cover 21 is fitted closely to the lens portion.

The endoscope cover fixing device 41, which constitutes a member for fixing the position of the cover on the endoscope 31, is mounted in proximity of a proximal end 23 of the cover 21.

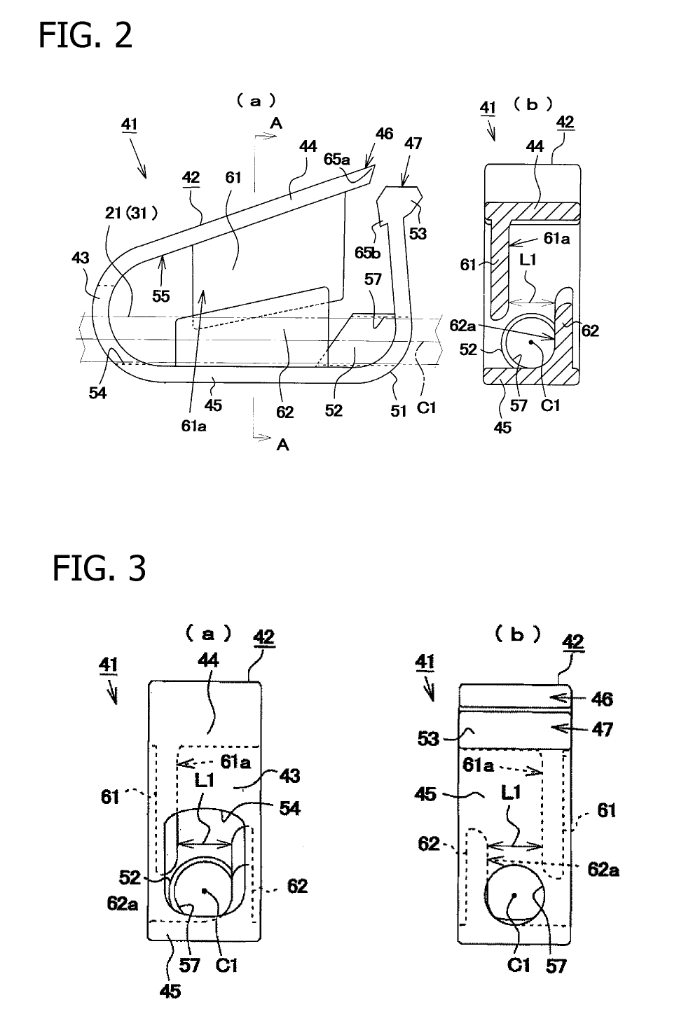

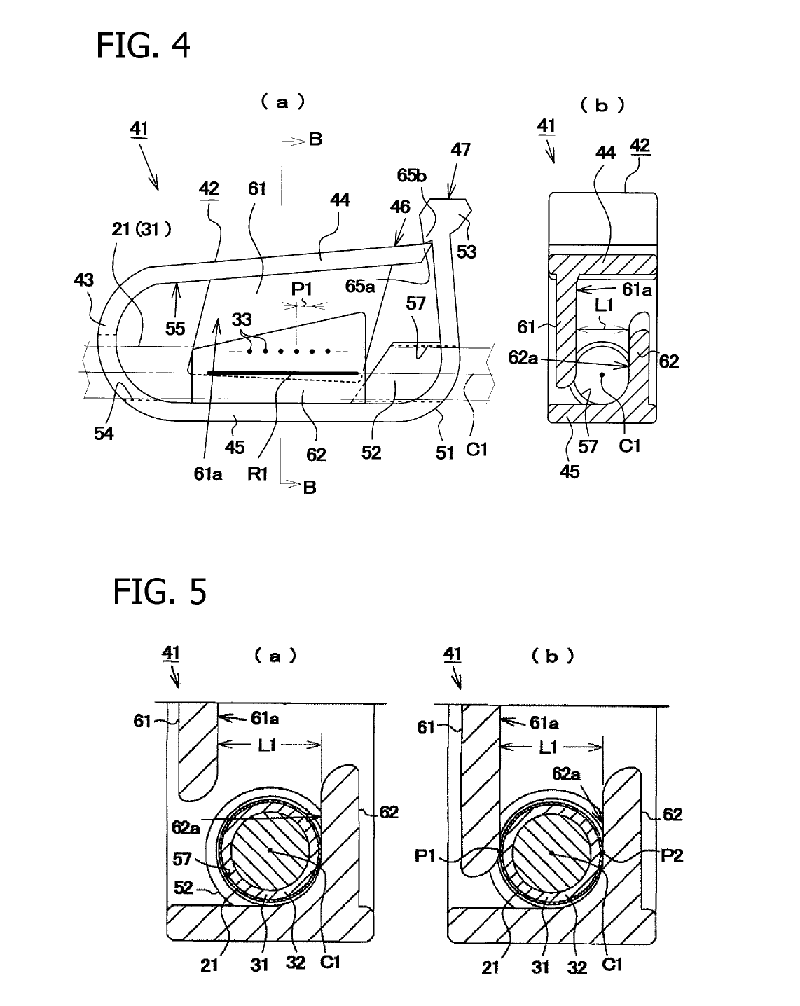

As illustrated in FIG. 1 and FIG. 2, the endoscope cover fixing device 41 includes a fixing device main body 42. The fixing device main body 42 is fabricated as a plate-like member of uniform width curved in an imperfect ring shape, though other forms are within the scope of the invention. The fixing device main body 42 includes a curved portion (bent section) 43 that bends at an angle of the order of 170° with respect to the side of the cover 21 comprising the distal end 22 as best illustrated in FIG. 1. One side of this curved portion 43 forms a first arm portion 44 while the other side forms a second arm portion 45. An additional curved portion 51 is provided in the second arm portion 45 that bends at an angle (approximately 90°, for example) with respect to the second arm portion. As illustrated in FIG. 4 and so on, the fixing device main body 42 is deformable in the direction in which the bend angle of the curved portion 43 increases, or in other words, in a direction in which the first arm portion 44 approaches the second arm portion 45.

In the open state shown in FIG. 2, a clearance exists between a first open end 46 of the distal end of the first arm portion 44 and a second open end 47 of the distal end of the second arm portion 45. The end portion of the first open end 46 is cut diagonally to form a first latch portion 65a. On the other hand, a latch release portion 53, preferably of greater thickness than other sections of the fixing device main body 42, is provided in the second open end 47. The region of the latch release portion 53 facing the concave surface 55 side of the fixing device main body 42 forms a second latch portion 65b able to lock with the first latch portion 65a. The role of the first latch portion 65a and the second latch portion 65b is to latch together to maintain the deformed state of the fixing device main body 42.

As shown in FIG. 2 and in FIG. 3, insert holes 54, 57 (insert portions) through which the endoscope 31 with the cover 21 placed thereover is insertable are formed in two parts of the fixing device main body 42. The proximal end side insert hole 57 is formed to penetrate the curved portion 51. A tubular portion 52 that protrudes to the concave surface 55 side is provided in the fixing device main body 42 in a position corresponding to the insert hole 57. The inner diameter of the insert hole 57 is slightly larger than the outer diameter dimension of the cover 21. The distal end side insert hole 54 is formed in such a way as to penetrate a curved portion 43. The aperture of the insert hole 54 is slightly bigger than the aperture of the insert hole 57 so that the endoscope 31, in a state in which it is covered by the cover 21, is easily inserted. The area of the aperture of the insert hole 54 may be even greater than illustrated.

As shown in FIGS. 2-5, the endoscope cover fixing device 41 comprises a pair of fixing protrusion portions 61, 62 that protrude from the concave surface 55 side of the fixing device main body 42. One fixing protrusion portion 61 is provided on the first arm portion 44, while the other fixing protrusion portion 62 is provided on the second arm portion 45. Notably, as best shown in FIG. 3, the fixing protrusion portions 61, 62 are offset in opposing directions along the width of the fixing device main body 42. In other words, the pair of fixing protrusion portions 61, 62 are mutually oppositely disposed in two regions on opposing sides of the concave surface 55. The expression “mutually oppositely disposed” used here refers to the arrangement of the protrusion portions 61, 62 to extend in opposing directions, and to their arrangement in positions that do not lie on the same plane.

Preferably, and as shown in FIGS. 2-5, the pair of fixing protrusion portions 61, 62 are planar members of equal thickness. The shape of the fixing protrusion portion 61 of the first arm portion 44 when seen from the thickness direction approximates a parallelogram. The fixing protrusion portion 62 of the second arm portion 45 is slightly smaller than the fixing protrusion portion 61 of the first arm portion 44 and describes a trapezoidal shape when viewed in the thickness direction. Notably, the interior of the distal end portion as defined by the pair of fixing protrusion portions 61, 62 is of a rounded shape designed to minimize damage to the cover 21 and the endoscope 31.

Flat contact surfaces 61a, 62a are formed in the interior of the pair of fixing protrusion portions 61, 62, and are disposed to describe a parallel positional relationship with a constant distance L1 therebetween. Similarly, when the endoscope 31 is inserted in the insert holes 54, 57, the flat contact surfaces 61a, 62a describe a parallel positional relationship to a longitudinal direction C1 of the endoscope 31. Notably, the aforementioned distance L1 is established in such a way as to be no less than the outer diameter dimension of the endoscope 31, and no more than the outer diameter dimension of the cover 21. In addition, the distance L1 is kept constant and does not change between the open state as shown in FIGS. 2 and 3 and the closed state as shown in FIG. 4.

The cover tube 32 of the endoscope 31 comprises a coil 33. The pitch P1 of the coil 33 in this embodiment is approximately 0.6 mm (see FIG. 4(a)), though other suitable values are within the scope of the invention. With these conditions in mind, the length of the pair of fixing protrusion portions 61, 62 along the longitudinal direction C1 of the endoscope 31 is formed in such a way as to be longer than the pitch P1 of the coil 33. In a preferred embodiment, the length of the pair of fixing protrusion portions 61, 62 along the longitudinal direction C1 of the endoscope 31 is established to be no less than ¼ of the total length of the endoscope cover fixing device 41, and at least several times that of the pitch P1. For this reason, a region R1 where the contact surfaces 61a, 62a contact the outer circumferential surface of the endoscope by way of the cover 21 extends linearly along the longitudinal direction C1. The length of the contact region R1 is desirably several times greater than the pitch P1.

The fixing device main body 42 of the endoscope cover fixing device 41 of this embodiment hence does not comprise a pair of clamping portions as seen in conventional medical clamps. In other words, this embodiment disposes of structures that flatten and deform the endoscope 31.

The endoscope cover fixing device 41 may be manufactured by injection molding or the like of, for example, a synthetic resin material. In an embodiment, the fixing device main body 42, the tubular portion 52 and the pair of fixing protrusion portions 61, 62 from which the endoscope cover fixing device 41 is constituted are formed as one piece of a synthetic resin material. Provided the material is flexible, there are no particular restrictions to the synthetic resin material which can be used for the endoscope cover fixing device 41, and various materials, most notably including polypropylene, polycarbonate and nylon, may be employed.

A method for using the endoscope cover fixing device 41 of this embodiment is described. Prior to the endoscope 31 being used, the cover 21 is placed over the endoscope 31. The inner surface of the distal end 22 of the cover 21 is disposed to fit closely against the lens portion. The fixing device 41 is prepared and setto the open state. The endoscope 31, in a state in which it is covered by the cover 21, is inserted into the insert holes 54, 57 through the distal sides thereof and the fixing device 41 is moved to a position in close proximity of the proximal end 23 of the cover 21 (see FIG. 1). Insertion of the endoscope 31 into the fixing device 41 may be performed prior to placement of the cover 21 over the endoscope 31. At this point, the endoscope 31 is disposed with respect to the fixing device 41 in a state in which it is covered by the cover 21 (i.e., it is disposed along the second arm portion 45).

With the fixing device 41 held in the fingers, pressure is applied thereto in such a way that the first arm portion 44 approaches the second arm portion 45. The fixing device main body 42 is caused to deform in a direction in which the bend amount of the curved portion 43 increases. Thereupon, the endoscope cover fixing device 41 of the open state as shown in FIGS. 2 and 3 is established in the closed state as shown in FIG. 4, and the first latch portion 46 and second latch portion 47 latch together to maintain the closed state. At this time, even though the pair of fixing protrusion portions 61, 62 move in parallel in mutually opposing directions, no marked change in the distance L1 between the contact surfaces 61a, 62a occurs (see FIGS. 5(a),(b)). With the distance L1 maintained to be constant in this way, the contact surfaces 61a, 62a contact opposing positions P1, P2 on the outer circumferential surface of the endoscope 31 by way of the cover 21, and this action completes the positional fixing operation.

Following the use of the endoscope 31, the latch release portion 53 of the endoscope cover fixing device 41 is operated to release the lock between the first latch portion 65a and the second latch portion 65b which, in turn, results in restoration to the initial unlocked state, or the open state. The fixing device main body 42 is elastically restored to its original shape, and the open state of the fixing device 41 as shown in FIGS. 2 and 3 is established. As a result, the fixing device 41 is then removable. While the used fixing device 41 may be discarded together with the cover 21, it may be cleansed and disinfected, and used again in accordance with need.

The following benefits, among others, are achieved by the embodiment described above. In the fixing device 41 of this embodiment in the configuration described above, even when the bend amount during the positional fixing operation increases, a constant pressure force is exerted on the endoscope 31 in the radial direction. In contrast, when a conventional medical clamp is employed and a provisional positional fixing is established, the cover and the endoscope are fastenably clamped by a pair of clamping portions thereof and then locked in the clamped state so established. Accordingly, a large pressure force is exerted in the radial direction on the endoscope. Using the fixing device 41 of this embodiment in the current invention, the exertion of excessive pressure force to the endoscope 31 in a state in which it is covered by the cover is avoided. As a result, the cover 21 is able to be reliably fixed in position on the endoscope 31 without associated damage.

Additionally, the contact region R1 afforded by the contact surfaces 61a, 62a of the fixing device 41 extends linearly along the longitudinal direction C1 of the endoscope 31. The length of this contact region R1 is established in such a way as to be larger than the pitch P1 of the coil 33 of the over-tube 32 of the endoscope 31. Accordingly, the pressing force exerted by the contact surfaces 61a, 62a is reliably dispersed rather than concentrated on a specific region of the endoscope 31. Accordingly, the damage to the endoscope 31 is able to be more reliably prevented.

### Second Embodiment

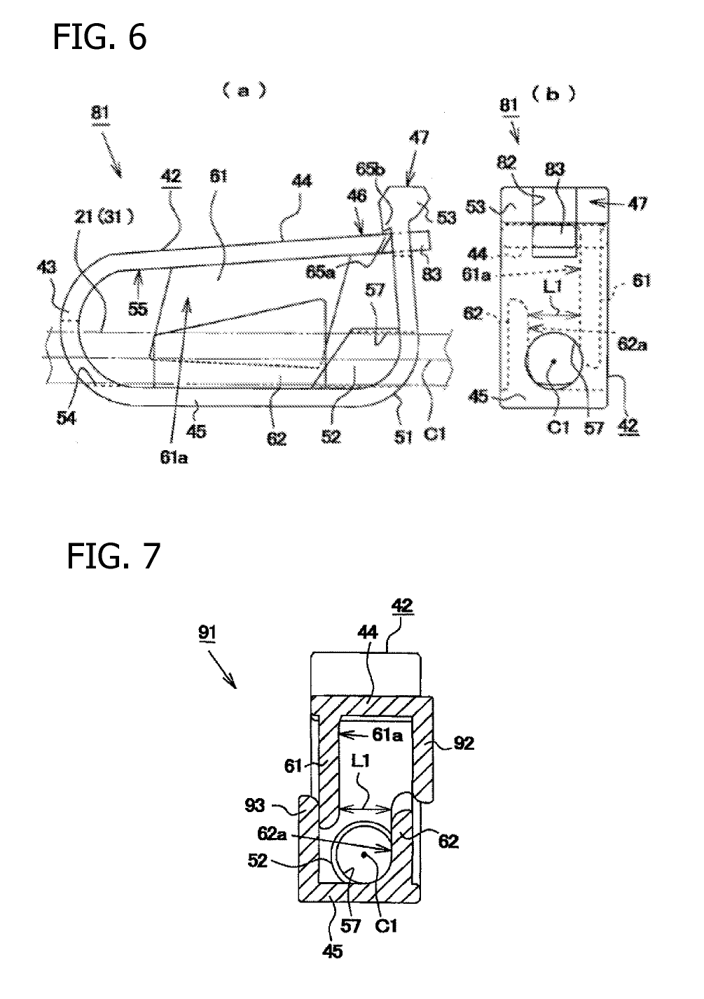

A fixing device 81 of a second embodiment of the present invention will be hereinafter described with reference to FIG. 6.

The fixing device 81 of this second embodiment differs from the first embodiment in that it additionally comprises a contact surface distance-maintaining mechanism. Specifically, a groove 82 from which the contact surface distance-maintaining mechanism is constituted is formed as a notch in the second open end 47 of the second arm portion 45 of the fixing device main body 42. A protrusion 83 from which the contact surface distance-maintaining mechanism is constituted is formed in the first open end 46 of the first arm portion 44 of the fixing device main body 42. The width of the protrusion 83 is slightly less than the width of the groove 82 and, as a result, the protrusion 83 can be inserted in the groove 82 during the deformation of the fixing device main body 42. As a result of the inability of the protrusion 83 to move in the width direction of the groove 82 at this time, displacement of the pair of fixing protrusion portions 61, 62 in the width direction of the fixing device main body 42 is prevented, and the distance L1 between the contact surfaces 61a, 62a is reliably maintained.

Accordingly, in this embodiment, a deformed state is maintained by the first latch portion 65a and second latch portion 65b, and an increase in the distance L1 between the contact surfaces 61a, 62a is prevented by the contact surface distance-maintaining mechanism. Because a constant pressing force is maintained during the positional fixing operation, the cover 21 is even more reliably fixed on the endoscope 31.

### Third Embodiment

A fixing device 91 of a third embodiment of the present invention will be hereinafter described with reference to FIG. 7.

The fixing device 91 of this third embodiment comprises a different type of contact surface distance-maintaining mechanism to the contact surface distance-maintaining mechanism of the second embodiment described above. A protrusion piece 92 from which the contact surface distance-maintaining mechanism is constituted is provided in a parallel, positional relationship to the fixed protrusion portion 61 in a position separate from the fixed protrusion portion 61 in the concave surface 55 side of the first arm portion 44. The inner surface side of this protrusion piece 92 is in slidable contact with the outer surface side of the other fixed protrusion portion 62. As a result, misalignment of the fixed protrusion portion 62 in the width direction is controlled. In addition, a protrusion piece 93 from which the contact surface distance-maintaining mechanism is constituted is provided in a parallel positional relationship to the fixed protrusion portion 62 in a position separate from the fixed protrusion portion 62 in the concave surface 55 side of the second arm portion 45 of the fixing device main body 42. The inner surface side of this protrusion piece 93 is in slidable contact with the outer surface side of the other fixed protrusion portion 61. As a result, misalignment of the fixed protrusion portion 61 in the width direction is controlled.

Accordingly, in this embodiment, the deformed shape is maintained by the first latch portion 65a and second latch portion 65b, and increase in the distance L1 between the contact surfaces 61a, 62a is prevented by the contact surface distance-maintaining mechanism. The maintenance of a constant pressing force during the positional fixing operation affords an even more reliable positional fixing of the cover 21 to the endoscope 31.

### Other Embodiments

Having described the invention in detail, it will be apparent that modifications and variations are possible without departing from the scope of the invention defined in the appended claims.

For example, while the bent section of the all embodiments is constituted from the curved portion 43, it may also be constituted from an elbow-shaped portion, or from other shapes.

The insert portion into which the endoscope 31 can be inserted in a state in which it is covered by a cover 21 is constituted by a hole that penetrates the fixing device main body 42. However, the configuration thereof is not limited in this manner and, for example, a notched portion or similar structure may also be adopted. The tubular portion 52 provided correspondence to the proximal end side insert hole 57, and may also or alternatively be omitted. In addition or in alternative, this tubular portion 52 may be provided corresponding to the distal end side insert hole 54.

While the aforementioned embodiments are produced as one piece of material by molding of the fixing device main body 42 and the pair of fixing protrusion portions 61, 62 from the same synthetic resin material, the pair of fixing protrusion portions 61, 62 may instead be molded separately from the fixing device main body 42 and joined by any suitable means (for example, adhesive-based adhesion or thermal welding). Although a synthetic resin material is selected as the material for moulding the fixing devices 41, 81, 91 of the aforementioned embodiments, a metal material such as stainless steel or aluminium may also be selected.

In other variations, a configuration in which the pair of protrusion portions 61, 62 of the fixing device are disposed in one of the two opposing regions of the concave surface 55 (i.e., either in the concave surface 55 side of the first arm portion 44 or the concave surface 55 side of the second arm portion 45) may be adopted. The parallel movement of the pair of fixing protrusion portions 61, 62 in the same direction as the direction in which the fixing device main body 42 is deformed allows the contact surfaces 61a, 62a to contact the aforementioned positions P1, P2 by way of the cover 21.

While the embodiments described above describe the employment of fixing devices 41, 81, 91 for fixing the position of the cover 21 on the medical endoscope 31, the application thereof in other medical instruments (including industrial endoscopes) is also possible.

Notably, the technical ideas derived from the embodiments of the present invention are outlined below.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above constructions, methods and products without departing from the scope of the invention, it is intended that all matter contained in the above description and shown in the accompanying drawings shall be interpreted as illustrative and not in a limiting sense.

What is claimed is:

1. An endoscope cover fixing device employed for, when a cover having a closed distal end is placed over an endoscope, fixing the position of a cover on the endoscope, said cover having a closed distal end and placed over the endoscope, said endoscope cover fixing device comprising:
a fixing device main body comprising a bent section that is deformable in a direction in which a bend amount thereof increases, and further comprising an insert portion into which said endoscope is insertable; and
a pair of fixing protrusion portions comprising a contact surface, said pair of fixing protrusion portions protruding from a concave surface side of said fixing device main body,
wherein said pair of fixing protrusion portions are mutually oppositely disposed in two opposing regions and on both sides of said concave surface,
wherein the contact surfaces of said pair of fixing protrusion portions are disposed in a parallel positional relationship with a constant distance therebetween and in a parallel positional relationship to a longitudinal direction of said endoscope when said endoscope is inserted in said insert portion,
and wherein said pair of protrusion portions move in parallel in opposing directions during deformation of said fixing device main body to ensure contact of said contact surfaces, by way of said cover, with opposing positions on an outer circumferential surface of said endoscope.

2. The endoscope cover fixing device according to claim 1, further comprising:
two open ends formed in said fixing device main body, said two open ends comprising latch portions which lock together to maintain a deformed state and
a contact surface distance-maintaining mechanism for preventing an increase in the constant distance between the contact surfaces in a width direction as a result of a displacement of said pair of fixing protrusion portions in said deformed state.

3. The endoscope cover fixing device according to claim 1, wherein a region across which said contact surfaces contact the outer circumferential surface of said endoscope by way of said cover extends linearly along the longitudinal direction of said endoscope, and a length of the region of contact is established to be larger than a pitch of a coil of an over-tube of the endoscope.

4. The endoscope cover fixing device according to claim 3, further comprising:
two open ends formed in said fixing device main body, said two open ends comprising latch portions which lock together to maintain a deformed state and
a contact surface distance-maintaining mechanism for preventing an increase in the constant distance between the contact surfaces in a width direction as a result of a displacement of said pair of fixing protrusion portions in said deformed state.

5. An endoscope cover fixing system for, when a cover having a closed distal end is placed over an endoscope, employing a fixing device to fix a position of the cover on the endoscope, wherein said endoscope cover fixing system comprises a pair of fixing protrusion portions that have a contact surface and a parallel positional relationship, wherein said contact surfaces contact opposing positions on a outer circumferential surface of said endoscope by way of said cover with a constant distance maintained between the contact surfaces and, as a result, a constant pressing force is exerted during said fixing.
